(19) United States
(12) Patent Application Publication
(10) Pub. No.: US 2007/0031447 A1
(43) Pub. Date: Feb. 8, 2007

(54) Method of isolating biologically active fraction containing clinically acceptable native S-lipopolysaccharides obtained from bacteria producing endotoxic lipopolysaccharides

(75) Inventors: Petr Gennadievich Aparin, Odintsovo (RU); Vyacheslav Leonidovic Lvov, Moscow (RU); Stanislava Ivanovna Elkina, Moscow (RU); Marina Eduardovna Golovina, Moscow (RU); Vladimir Igorevich Shmigol, Moscow (RU)

Correspondence Address: LADAS & PARRY, 26 WEST 61ST STREET, NEW YORK, NY, 10023, US

(73) Assignees: PETR GENNADIEVICH APARIN; VYACHESLAV LEONIDOVICH LVOV

(21) Appl. No.: 11/494,755

(22) Filed: Jul. 27, 2006

Related U.S. Application Data
(62) Division of application No. 10/445,461, filed on May 27, 2003, now abandoned.

(30) Foreign Application Priority Data
Jun. 26, 2002 (RU) 2002117055

(51) Int. Cl. A61K 39/02; C07K 14/25

(52) U.S. Cl. 424/190.1; 530/350

(57) ABSTRACT

A biologically active fraction (BAF) is presented containing mainly S-lipopolysaccharide (LPS) from gram-negative bacteria producing endotoxic LPSs. These fractions are characterized in that in the lipid A of S-LPS the mole ratio of D-glucosamine and β-hydroxy acids selected from the group comprising β-hydroxydecanoic, β-hydroxydodecanoic, β-hydroxytetradecanoic, β-hydroxyhexadecanoic, is approximately 2:1-4, and the mole ratio of D-glucosamine and higher fatty acids, connected both by amide and ester links, in the lipid A of S-LPS is approximately 2:3-7. A method of isolating BAF, containing mainly S-LPS from gram-negative bacteria producing endotoxic S-LPSs, is also presented. The obtained BAFs have pyrogenicity at the level of commercial polysaccharide vaccines and low endotoxicity, have high immunogenicity, which makes it possible to use them as vaccines for mammals, including humans. They are an inducer of cytokines and also may be considered to be a prophylactic tolerogenic anti-shock preparation.

### FIELD OF THE INVENTION

The invention relates to the field of medicine, in particular, to a method of isolating a biologically active fraction (BAF), containing mainly native low-toxic S-lipopolysaccharide (S-LPS) obtained from bacteria producing endotoxic lipopolysaccharides (LPSs), for use in clinical and experimental medicine for the purpose of prophylaxis and treatment of diseases.

### BACKGROUND OF THE INVENTION

LPSs are the main polysaccharide antigens of gram-negative bacteria. They are located on the external surface of the outer membrane of a cellular wall and play the most important role in the pathogenesis of many infections (1). LPSs actively participate in the building and functioning of a physiological membrane of a microorganism and are extremely important for its growth and viability (1, chapter 2). On the other hand, LPSs are the primary target for interaction with antibacterial drugs and components of the immune system of a host organism.

The molecule of LPS includes a hydrophilic heteropolysaccharide (O-specific polysaccharide), built from repeating oligosaccharide units and which basically determines the immunological specificity of the bacterial cell. O-specific polysaccharide covalently links to a branched “core” oligosaccharide comprising 10-12 monosaccharide residues, which in turn is coupled to a hydrophobic lipid fragment—lipid A, holding the LPS molecule in the outer membrane of the microbial cell. Three residues of 2-keto-3-deoxyoctulose acid (KDO), the ketosidic linkage of which is extremely acid-labile, are positioned on the border between the “core” and lipid A moieties. LPSs, which comprises all three fragments—O-specific polysaccharide, “core” and lipid A, are isolated from strains of gram-negative microorganisms, which on cultivation on solid cultural grown media in the form of smooth colonies and which, therefore, are called S-LPSs. LPSs lost the O-specific polysaccharide chain so-called R-LPSs are usually isolated from strains which grow in the form of rough colonies.

As distinction from an O-specific polysaccharide, which by itself is immunologically inactive, the lipid component of an LPS determines the whole complex of pathophysiological properties of an LPS, in particular, its high endotoxicity and pyrogenicity. Lipid A moiety also determines the adjuvant properties of LPSs and, as a result, their extremely high immunogenicity.

LPSs have a wide spectrum of “beneficial” biological characteristics, which are intensively studied in experiments on animals and humans, and also in vitro. The wide spectrum of “positive” biological properties is usually understood to mean the capability of LPSs to manifest themselves as powerful protective antigens, to exert antibacterial and antiviral activity, and also to act as an immunostimulator and adjuvant (1).

Immunization of animals with microgram amounts of LPS causes induction of high titers of specific antibodies of different classes, which determines their vaccine potential. On the other hand, recognition of LPS by macrophages results in the induction of many proinflammatory mediators, including the a tumor necrosis factor (α-TNF), interleukines 1, 6 and 12 (IL-1, IL-6 and IL-12, respectively), interferons (IFN) α and β, chemokines and lipid mediators (1). Furthermore, macrophages, activated LPSs may act as antigen-presenting cells for CD4⁺ T-cells. In cooperation with IFN-γ LPS stimulates macrophages for transcription of genes encoding NO-synthetase, which in turn results in synthesis of the toxic oxidant—NO. Stimulation of macrophages with the aid of LPS and IFN-γ results in the appearance of a highly-active phenotype, which is characterized by a powerful bactericidal and antitumoral potential (1, chapter 63). Thus, stimulation of macrophages by LPS results in the development of local immunity to gram-negative bacteria and, by means of the production of IL-1, IL-6 and α-TNF, to different remote and systemic effects, which plays an important role in the struggle not only against infections, but also against tumors.

On the other hand, native traditional LPSs are biologically active substances, which are very dangerous for a human. The getting into the organism of a small amount of LPS included in the composition of a bacterial product or as a contaminating substance may result in endotoxic shock, accompanied by severe hemodynamic disorders (1, chapter 55). However, useful pharmacological effects, manifested by LPS in quite a number of pathological states (infection, tumoral process), are so important that there are attempts made to use LPSs or derivatives thereof in clinical medicine in different directions (1, chapter 63).

The current approaches for the preparation of clinically applicable LPSs are related to partial modification of their structure, i.e. to change of some biological properties.

One of such approaches is chemical modification of LPSs, for example, mild alkaline or enzymatic treating resulting in changes in the structure of lipid A. The subsequent elimination of a portion of fatty acids and phosphate-containing substituents results on the one hand to a reduction of the endotoxicity of LPS, but on the other - to a reduction of the level of immune response right down to the complete loss of some immunobiological functions of modified LPSs.

Prior to the development of the group of compounds in accordance with the instant invention, it was not possible to directly use native LPSs as a vaccine because of their high toxicity. Therefore, some of the conjugated vaccines developed at the present time against infections caused by Salmonella enterica sv paratyphi A, Shigella sonnei, Shigella flexneri, tested on volunteers (2, 3, 4), include conjugates of the O-specific polysaccharide component of LPS with a protein carrier. The immunogenic potential of LPS, which is lost as a result of lipid A elimination, is partially restored by use of a protein carrier and optimization of the schemes of immunization, which are directed to the induction of a secondary immune response.

Another direction of clinical application of denaturated LPSs is tolerogenic anti-shock vaccines. The idea of using LPSs as a tolerogenic antishock vaccine in order to create tolerance the organism to subsequent massive entry of an endotoxin is not novel (1, chapter 50). At the same time, this approach does not lose its actuality in view of the extreme importance of control of clinical situations resulting in endotoxic shock. Recently in clinical and experimental studies the induction of “early” or “late” tolerance to subsequent administration of endotoxin not only by LPS, but also it's derivative monophosphoryl-lipid A (MPL), and also synthetic analogs of lipid A, was demonstrated (1, chapter 50). MPL is also well-known as an immunomodulator of a wide spectrum of action and as an adjuvant.

Another way to reduce endotoxicity of LPSs is the functional detoxification of LPSs by cyclic peptides (1, chapter 23), specific proteins (1, chapters 19, 21) or polycationic compounds (1, chapter 11), wherewith each of the compounds listed above is capable of specifically binding to a lipid A moiety LPS. This trend is regarded as promising for the purification of blood or plasma of patients in systems of extracorporeal treatment, using a solid-phase matrix comprising ligands specific for lipid A. However, at present which processes may occur when LPSs complexes with lipid A ligands are introduced into the organism of an animal have been insufficiently studied. So, the question of their immunogenicity, biodegradability, toxicity, possibility of accumulating in different tissues, etc., remain unclear.

The experience of using LPS for cancer treatment requires separate consideration. Clinical studies concerned to the application of LPS for tumor therapy are well known (1, chapter 63). At present not only all possible variants of modified LPSs (chemical derivatives, synthetic analogues, products of functional detoxification), but also native highly-toxic endotoxin are used for treatment of tumors. The latest clinical studies in this direction are directed to the study of the possibilities of combined LPS and γ-interferon therapy. Due to high endotoxicity, it was only possible to use extremely low doses of LPSs for intravenous administration—1-4 ng per kg of weight.

As distinctive over traditional LPSs, the group of low-endotoxic native BAFs of LPS, which are presented in the invention, are distinguished by an extremely wide interval of doses for clinical use, which creates new possibilities for doctor creation of effective schemes of treatment.

### SUMMARY OF THE INVENTION

Our proposed original approach to get obtaining of BAFs of low-endotoxic native S-LPSs is a combination of modern and effective methods, upon use of which the possibility appears, on the one hand, of obtaining highly purified LPS (the content of impurities not more than 4%), and on the other hand—of isolating a low endotoxic fraction from the sum “parent” LPS, wherewith the conditions of carrying out all steps of isolation and purification presume maximum maintenance of the primary LPS structure.

The isolation and purification of the claimed BAFs include the extraction, enzymatic treatment of the extract to destroy foreign nucleic acids and proteins and the subsequent fractionation of raw LPS by different methods.

The main method at the stage of extraction is treatment with a 45% hot aqueous phenol by Westphal (5). At the present time this method in particular is used for the isolation of LPS, since it, on the one hand provides a high yield of LPS, and on the other hand—presumes stability of glycosidic and ketoside links of monosaccharide residues and links of non-carbohydrate substituents in the polysaccharide and lipid fragment of LPS.

Enzymatic treatment of the extract is carried out for decomposition ribo- and deoxyribonucleic acids and also protein components of a bacterial cell. Enzymatic hydrolysis of nucleic acids is carried out by the simultaneous action of ribonuclease-A and deoxyribonuclease in buffers containing calcium and magnesium ions. The selection of the nuclease is not principle in character: when an enzyme with reduced activity is used it is necessary to increase its concentration and reaction time. Further, a disodium salt of ethylenediaminetetraacetic acid is added to the reaction mixture to couple the ions of bivalent metals and protease, usually proteinase K.

The isolation of BAFs of S-LPSs from unpurified LPS obtained after treatment with enzymes may be carried out by an extraction mixture of chloroform-methanol-0.2M HCl [6]. However, when a mixture with the ratio of extractants indicated in the paper [6] is used for extraction, a product is isolated with pyrogenicity which exceeds by 8-16 times the limit established by Pharmacopedae for polysaccharide vaccines (0.05 μg/kg of rabbit weight).

In view of this the ratio of the solvents in the mixture for extraction was changed as compared with the ratio described in the paper [6] in order to enhance its polarity and hydrophilicity and became 1:1:0.4-0.5. Wherewith the yield of apyrogenic BAF from S-LPS did not exceed 1% of the weight of the unpurified LPS.

Ultracentrifugation is carried out in aqueous, possibly buffered, solutions, possibly containing chaotropic agents, with a concentration of LPS which no more 1%. The conditions for ultracentrifugation, including the speed and time, are specially selected for an LPS isolated from each concrete microorganism. So, when the speed and/or time of ultracentrifugation is reduced, an increase of low-molecular LPS content in the final product is observed, and when these parameters increase—there is a sharp reduction of the yield. A criterion of the optimum selection of the conditions for ultracentrifugation may be the level of endotoxicity of the BAF isolated from the supernatant.

It is important to underline that ultracentrifugation, and also all the methods for isolating apyrogenic BAF from S-LPS, described below, may be used for both a product of enzymatic processing and a product obtained at the stage of extraction by a mixture of chloroform-methanol-0.2M HCl.

Fractionation of BAF by using preparative electrophoresis in a polyacrylamide gel appears to be relatively promising, since the possibility for isolating sufficiently narrow LPS fractions, varying the number of cross-links in the gel (from 7.5 to 12.5%), appears, but, in turn, it has a number of drawbacks. Thus, there are obvious difficulties in the separation of the gram amounts of the initial LPS, and also complications related to the possibility for contamination of the desired product with fragments of the degradation (mechanical or chemical) of the polyacrylamide gel.

The drawbacks indicated above are not present in the method of fractionating LPS with the use of ultrafiltration membranes of the “Millipore” firm. However, because of significant differences in the size of the pores of these membranes, only sufficiently “wide” fractions of S-LPS may be isolated as a result of fractionation, for which fractions the interval of values of the molecular weight may reach 20-30 kDa, i.e., in the case of using, for example, membranes with a “cut off” of 50 kDa, molecules of S-LPS with a molecular weight of 60 or even 70 kDa may pass through the pores. Furthermore, in some cases, in order to enhance the quality of separation it becomes necessary to use detergents, such as, for example, Triton X-100, the role of which is to remove LPS micelles. In that case the removal of the detergent from the final product becomes a special problem. In order to do this, lower alcohols, usually ethanol in a concentration of up to 30%, are often added to the LPS solution during fractionation. Wherewith, the use of this approach is most convenient for the removal of low-molecular fractions from nonfractionated LPS.

Ion-exchange gel-chromatography is a sufficiently convenient method of separation of S-LPSs only when they contain O-specific polysaccharides including charged residues, for example, in the case on the fractionation of LPS Sh. sonnei, phase 1 (O-polysaccharide carries carboxyl and amino groups) (7).

The main difficulty during the use of both ion-exchange and gel-penetrating chromatography is the marked tendency of LPS to aggregation. In order to eliminate the effect of this undesirable factor, it is possible to include organic solvents (alcohols, acetonitrile), chaotropic agents or detergents (nonionic in the case of ion-exchange chromatography) into the eluent. In spite of the vast choice of commercially available gels, DEAE-Sephadex is usually used for ion-exchange chromatography and Sepharose 2B, 4B or 6B-CL—for gel-penetrating chromatography.

One of the most convenient methods for separating amphiphilic high-molecular compounds is hydrophobic chromatography with the use of butyl-, octyl- or phenyl-Sepharose. We showed that electrodialysis with the subsequent formation of a triethyl ammonium salt of LPS get the preparation of a sample for preparative hydrophobic chromatography [7] may be successfully replaced by treatment with chalating ion-exchange resins. Attention should also be directed to the fact that the sum hydrophobicity of an LPS molecule determines not only the lipid component, but also, to a significant degree, the nature of the monosaccharides included from which the O-specific polysaccharide is built (the presence therein of O— and N-acetyl-, and also desoxysacchars). On the other hand, the wide choice of matrixies and eluents for gradient separation makes it possible in many cases to successfully optimize the process of isolating the desired fraction of S-LPS.

Thus, a methodology for isolation of the claimed BAF from different gram-negative bacteria is proposed, which includes as obligatory steps the extraction, with subsequent degradation, of foreign proteins and nucleic acids by the action of enzymes, while further fractionation in order to obtain the desired product may be carried out with the use of one of the methods described above or combination thereof. Wherewith, in each concrete case several factors have an effect on the choice of the fractionation method, in particular, the species of the microorganism, the desired set of immunobiological characteristics (for example, pyrogenicity, toxicity), the yield of BAF, the cost and characteristics of production and other factors.

The scheme presented above for isolating and purifying the claimed BAF on the base of S-LPS (as distinctive from modified LPSs preparations) presumes the maintenance of “nativity” (native mature of the LPS molecule) and “non-alteration” of the LPS primary structure.

Analyzing the composition and structure of the claimed BAFs, it should be noted that on the basis of data of the NMR spectra, in all the cases the structure of the O-specific polysaccharide component of the LPS, included in the composition of the claimed BAF, did not differ from that described earlier in literature.

With regard to the oligosaccharide “core” located in the LPS at the border between the O-specific polysaccharide and the lipid A, a study confirming its presence in the composition of the claimed BAFs was carried out with the aid of identification therein of two “obligatory” for the “core” components—a heptose [by the method of combined gas-liquid chromatography-mass-spectrometry (GLC MS)] and KDO (by a calorimetric reaction with thiobarbituric acid).

The only structural distinctions detected at present between “parent” LPSs and the proposed BAF preparations were established by us as a result of comparative analysis of the lipid A fatty acid composition by GLC-MS method.

Therefore, special attention was given within the frame of the present work, in particular to the lipid component of the claimed BAFs, since that component, as indicated above, determines the whole complex of pathophysiological properties of the LPS. Some specificities of the lipid component of the claimed BAFs will be given consideration below after a brief review of the structures of lipid A from the LPS of the Enterobacteriaceae family.

At the base of lipid A lies the disaccharide β-(1′-6)-D-glucosaminyl-D-glucosamine, which carries two phosphate groups—one in position 4′ of the unreduced residue of D-glucosamine, the other (connected by α-bond)—in position 1 of the reduced residue of D-glucosamine. Both amino groups of the aminosugar residues in the disaccharide are substituted by residues of (R)-3-hydroxytetradecanoic acid [14: O (3-OH)], while two other residues of this acid are connected by an ester bond to positions 3 and 3′ of both residues of D-glucosamine of the disaccharide fragment. Both residues [14: O (3-OH)], localized in the unreduced residue of D-glucosamine, in turn are substituted by residues of tetradecanoic and dodecanoic acids. This so-called “classical” structure of lipid A is characteristic (with some insignificant variations in the nature of the substituents in the case of phosphate groups) for the overwhelming majority of LPSs of the Enterobacteriaceae family.

A large number of publications in the last years are concerned to establishing the primary structure of lipid A of LPSs isolated from microorganisms of that and other families and genuses of gram-negative bacteria, their synthesis and synthesis of their multiple analogues, and also to a study and comparison of biological characteristics of isolated and synthesized lipids (1, chapters 14, 46, 47). The conclusion that any significant deviation from the “classical” structure of lipid A results in a marked and in some cases to a dramatic reduction of their endotoxicity is made from an analysis of the results of these studies (1, chapters 46, 47). Wherewith the qualitative and quantitative composition of the fatty acids is the most important feature. So, lipid A, which does not contain the “classical” set of six fatty acids but rather has seven or five residues thereof in its composition, exhibits significantly lower in vivo endotoxic activity [pyrogenicity (rabbits) and lethal toxicity (mice)] (1, chapters 46, 47) as compared with the “classical” lipid A. Furthermore, as distinctive from the “classical” lipid A, characteristic for the lipids A indicated above are the presence of fatty acids with a longer or shorter hydrocarbon chain, which may contain an HO group at C-2 (and not C-3), an oxo group, a double bond, branching at the methylene link adjacent to the methyl group, and even a cyclopropane fragment. Thus, it is shown that low LPS endotoxicity of Bacteroides fragilis is connected with the fact that the lipid A of these LPSs has a unique set of fatty acids: a portion of the fatty acids with tetradecanoic skeleton is replaced by fatty acids with sixteen carbon atoms (1, chapter 7).

It was shown within the frame of the present study that the lipid A of the claimed BAF together with “obligatory” fatty acids (in particular, [14:0 (3-OH)], tetradecanoic and dodecanoic acids) also contained unusual fatty acids, for example hexa- and octadecanoic, wherewith a sharp decrease of the [14:0 (3-OH)] content is also observed. Consequently, the structure of lipid A of the claimed BAFs on the basis of S-LPS significantly differs from the “classical,” which clearly may have an effect on their endotoxic characteristics.

In view of this, it becomes possible to state that highly endotoxic LPSs of gram-negative microorganisms may contain fractions of S-LPS with a reduced level of endotoxicity and pyrogenicity, which may be used in clinical practice.

In this study we obtained several proofs of the “nativity” of the claimed preparations. In the first place, a comparison of the fatty acid composition of lipid A and the NMR spectra of polysaccharide fragments of BAFs isolated by different methods (for example, by the action of aqueous phenol and trichloroacetic acid) has shown their complete identicalness in respect to these characteristics. In the second place, pronounced serological reactions between BAF and corresponding anti-O-serums and also serums of patients (with a bacteriologically confirmed diagnosis) were observed in all cases. In the third place, in the case of the most widely spread method of isolating LPS—extraction by hot aqueous phenol, it was shown that with repeated processing of the isolated preparations under conditions of extraction neither changes of the fatty acid composition nor of the spectral characteristics of the isolated preparations take place. In the fourth place, all the BAFs exhibited high immunobiological activity.

The BAFs protected by the present invention in respect to the level of safety significantly differ from LPS preparations obtained by traditional methods. They satisfy the requirements of the Technical Committee of Experts of the WHO in respect to the safety parameters for meningococcus and typhoid Vi-antigenic polysaccharide vaccines (8).

After parenteral administration of BAF from Sh. sonnei to volunteers in doses of 25, 50, 75 μg under the control of the National Control Authorities of Russia (NCA) within the frame of clinical tests of dysentery vaccine, not only endotoxic shock but also significant general or local endotoxic reactions were not observed. Thus, the doses of clinical usage of the claimed BAFs are comparable with such for capsular vaccine polysaccharides and significantly (by 100-1000 times) higher than doses of routine LPSs for administration to a human during different clinical studies (9). Many subsequent clinical tests of the preparation confirmed its high level of safety: the absence of general reactions such as chill, headache, only a slight ( <37.6° C.) rise of temperature in not more than 10% of those vaccinated, very rare local reactions in the form of reddening or pain at the point of administration.

BAF from Sh. sonnei was also safe for administration to children of different age groups—3-6, 7-10, 10-14 years old, in a dose of 25-50 μg subcutaneously. The level of reactogenicity, evaluated by general and local reactions, does not significantly differ in the child and adult contingents.

The safety of BAF, the absence of pyrogenic reactions in the case of subcutaneous administration of vaccine doses of the preparation in clinical and experimental studies, may be explained by the relatively low activation by BAF of α-TNF (one of the key mediators of the endotoxic reaction) production. The subcutaneous administration of BAF from Sh. sonnei to volunteers in doses of 50, 75 μg does not result in a reliable increase of the a-TNF content in the blood serum of volunteers after primary or secondary immunization.

The experimental and clinical studies carried out also show that the claimed BAFs are powerful immunogens and may be used as vaccines. They cause the induction of O-specific protective antibodies in animals, including humans, wherewith the humoral immune response is presented by all the main classes of antibodies IgG, IgA, IgM. Such antibodies are bactericidal for the microorganisms against which they are directed. On models of anti-infective immunity they provide protection of a macroorganism against the homological strain of a typhoid or shigellosis pathogen (10). In the case of immunization of volunteers by separate BAF preparations, an expressed rise of the level of serum O-specific antibodies and seroconversion (rise of the level of antibodies ≧4 times) in 80-90% of immunized persons is observed. The persistence of high levels of specific antibodies results in protection of the population against Sonne shigellosis infection.

A characteristic feature of the immunogenicity of low-endotoxic BAFs is the powerful multisystem activation of both systemic and local IgA immune response. The immunization of guinea pigs with BAF from Sh. sonnei results in the induction of local immunity and protects their mucus of the eye conjunctiva against infection by a homologous virulent strain. 0-specific antibodies produced locally and detected with the aid of MAT to α-chains and secretory component are determined in the saliva of immunized volunteers.

Field trials of vaccine carried out by the NCA of Russia against Sonne shigellosis, which vaccine is a pharmaceutical composition based on BAF, in an infection endemic area of the Saratov region, showed high prophylactic efficacy of the preparation. The preparation provided protection of the civilian population in the most unfavorable summer-autumn period of rise of the Sonne shigellosis incidence. The average index of efficacy of the vaccine exceeded 90% (12).

The use of low-endotoxic BAFs as a tolerogenic antishock vaccine is effective and may remove the problem of toxicity of a similar preparation, which is very important for patients of a surgical clinic. The complete viability of experimental animals after injections of BAF from LPS of Shigella sonnei, Salmonella enterica sv typhi, Escherichia coli 12 hours prior to the subsequent administration of a lethal dose of endotoxin provides proof of the formation of an expressed immunity against bacterial endotoxin.

Low endotoxic BAFs have a many-sided action on the immune system and are immunomodulators which have an effect on the resistivity of a macroorganism to tumoral growth. The immunomodulating action of BAFs as distinctive over vaccine action is especially clearly manifested at higher doses of the preparations—50 - 150 μg and multiple administration.

The group of claimed preparations has an anticancerogenic effect that has been established in an experiment in vivo with use of inoculated cells of mastocytoma P855. Two-time administration of a preparation of BAFs from Salmonella enterica sv typhi to Balb/c mice prior to inoculation of mastocytoma cells results in the growth of viability of animals. A similar effect was noted with the administration of the preparation in early (to 24 hours) periods after inoculation.

The BAF from LPS significantly enhances the resistance of mice to infection with the natural infection Salmonella enterica sv typhimurium—a bacterial pathogenesis with obligatory intracellular parasitization.

The main immunomodulating effect of the group of claimed preparations is rational activation of cytokines, mediating the mechanism of anti-infective immunity. Among the key immunity mediators which play an important role in immunity against viral and bacterial infections with intracellular parasitization, the most important is γ-interferon. The BAF from the LPS of Shigella sonnei is a powerful inducer of γ-interferon in vivo in a dose of 100 μg.

The BAFs proposed in the instant invention may be used not only as vaccines, but also as vaccine matrix for the construction of conjugated vaccines for mammals, including humans. A conjugate of a capsule polysaccharide of vaccine quality S. typhi (Vi-antigen), with BAF from Sh. sonnei selected as the carrier, was obtained with the use of conjugating agents, in particular carbodimide. The conjugates, bound by a strong amide binding, were stable under dissociating conditions. A BAF may also be used as a matrix for protein antigens, which is especially important because of the virtually complete absence of such matrixes in vaccinolology.

The possibility of vaccines and immunogens construction on the base of the proposed BAFs without creating covalent chemical bonds between polymeric molecules should be underlined. We have established the fact that the composition of high-molecular micelles formed during the self-assembly of BAFs includes a model protein antigen—human serum albumin. Such “quasi-conjugated” vaccines are high-molecular 1000-5000 kDa complexes including molecules of the necessary antigens and micelle-forming immunostimulating BAF carrier.

Separate consideration should be given to the question concerning the architectonics of the supermolecular structures of LPS, which exist in the outer membrane of the cell wall of gram-negative bacteria and are obviously destroyed in the process of extraction, but are restored again after removal of the polymer and low-molecular bacterial components in the process of purifying the LPS. The isolated LPSs in aqueous solutions tend to self-organize into structures which, as shown with the aid of electronic microscopy (1, chapter 11), remind one of fragments of a bacterial membrane.

Thus, the group of proposed preparations is highly immunogenic clinically applicable BAFs containing primarily S-LPSs with a low level of endotoxicity and pyrogenicity, which includes lipid A, the fatty acid composition of which may differ from the fatty acid composition determined for the starting sum LPS of the corresponding microorganism.

### DESCRIPTION OF THE DRAWINGS

The products obtained in accordance with the present invention were subjected to analysis with the aid of NMR-spectroscopy and chromato-mass spectrometry analysis.

FIG. 1 shows the spectrum ¹³C-NMR of O-specific polysaccharide Sh. sonnei.

FIG. 2 shows the spectrum ¹H-NMR of O-specific polysaccharide Sh. sonnei.

FIG. 3 shows a chromatogram of methanolysate of lipid A, isolated from BAF of Sh. sonnei.

FIG. 4 shows a chromatogram of methanolysate of lipid A from LPS of Sh. sonnei, isolated by the Westphal method.

### EXAMPLES

The following examples, presented in order to concretize and illustrate the invention, do not have the object of limiting the claims of the present invention.

### Example 1

1. Extraction

Extraction of bacterial cells Sh. sonnei, phase 1 (20 g), dried by acetone, was carried out by 45% aqueous phenol (700 ml) with intensive mechanical stirring in a thermostat vessel at 68-72° C. during 15 minutes, the suspension cooled to 10-15° C. was subjected to centrifugation for 40 minutes at 5000 g, the upper aqueous layer was isolated and dialyzed for 5 days against distilled water, the undissolved residue was removed by centrifugation at 13000 g and the supernatant was lyophilized. The yield of the extraction product was 12% of the weight of dry cells. The content of nucleic acids (UV-absorption) and proteins (Laury method) was 35-40% and 10-15%, respectively. The extraction product thus obtained (initial concentration 100 μg/ml) was active in a reaction of inhibiting passive hemagglutination (RIPHA) with rabbit serum, obtained with immunization by a dead culture of Sh. sonnei, phase 1, in a dilution of 1:128.

II. Enzymatic Hydrolysis

Two grams of raw extract containing mainly LPS, nucleic acids and water-soluble proteins were dissolved in 100 ml of a buffer containing 0.2 M NaCl, 0.05 M TRIS-HCl and 0.001 M MgCl₂ and CaCl₂, pH =7.2-7.6, the solution was stirred for 2 hours at a temperature of 37° C. with ribonuclease A (activity 50-100 units/mg, 50 μg/mI of solution) and desoxyribonuclease (activity 400-800 units/mg, 5 μg/ml of solution), then proteinase K (activity 10-20 units/mg, 20 μg/ml of solution) was added to the solution, the solution was stirred for 1 hour at a temperature of 50° C. The reaction mixture was dialyzed for 72 hours against distilled water and lyophilized.

III. Ultracentrifugation

One gramm of the product obtained in step II was dissolved in 120 ml of water, ultracentrifugation of the solution was carried out with use of the Bekman ultracentrifuge (U.S.A.) at a temperature of 5° C., acceleration of 80,000 g for 8 hours. The obtained supernatant was lyophilized.

IV. Isolation of BAF with the Aid of Extraction

If necessary, instead of step III, extraction was carried out by a mixture of chloroform-methanol-0.2M aqueous HCl. In order to obtain BAF from Sh. sonnei, the mixture indicated above was used with a ratio of the components 14:13:5; the content of the solid phase was 10 mg/ml.

V. Fractionation on Porous Matrixes

If necessary, instead of step III and/or IV, a preparative electrophores in a 10% polyacrylamide gel was used (thickness of the gel 1.5 mm, movability of BAF relative to bromphenol blue 0.1-0.3; elution was carried out with the aid of Whole Gel Eluter, Bio-Rad) or hydrophobic chromatography (column with octyl-Sepharose in a buffer containing 10% propanol and 0.05M ammonium bicarbonate, pH=8.1) or with the aid of gel-chromatography on a column with Sephadex G-50 (1.5 M aqueous ammonium bicarbonate) or ultrafiltration with the aid of a Pellicon system (Millipore) (membrane with “cut-off” 10 kD) or ion-exchange chromatography on a column with DEAE cellulose (0.01-0.75M gradient of ammonium bicarbonate).

VI. Purification of BAF

Desalinization of BAF after fractionation, if necessary, was carried out by gel-chromatography on a column with sepharose 4B-Cl in water or 0.05M pyridine-acetate buffer at a temperature of 4° C., fractions containing BAFs, which were eluted near the void volume of the column, were combined and lyophilized.

TABLE 1
Isolation of BAF from unpurified LPS of Sh. sonnei using different
fractionation methods

| | | Yield of apyrogenic (in dose of |
|---|---|---|
| | | 0.05 μg/kg weight of rabbit) |
| Step | Method | BAF in % of weight of raw LPS |
| III | Ultracentrifugation | 3-5% |
| IV | Extraction of LPS by mixture of | <1% |
| | chloroform-methanol-0.2M HCl | |
| V | Preparative gel-electrophoresis | 1-3%* |
| | in polyacrilamide gel (PAGE) | |
| V | Ultrafiltration on “Millipore” | 5-7%* |
| | membranes | |
| V | Ion-exchange chromatography | <1% |
| V | Hydrophobic chromatography | 3-5% |
| V | Gel-chromatography | 3-5% |

*the dose reduced to 0.0125-0.0250 μg/kg weight of the rabbit because at a higher dose the product is pyrogenic.

### Example 2

Confirmation of the structure of O-specific polysaccharide and lipid A, which are isolated from BAF from Sh. sonnei, using NMR-spectroscopy and chromato-mass-spectrometry.

O-specific polysaccharide and lipid A were isolated from BAF from Sh. sonnei as a result of mild acid hydrolysis and subsequent deposition of unsoluble lipid A.

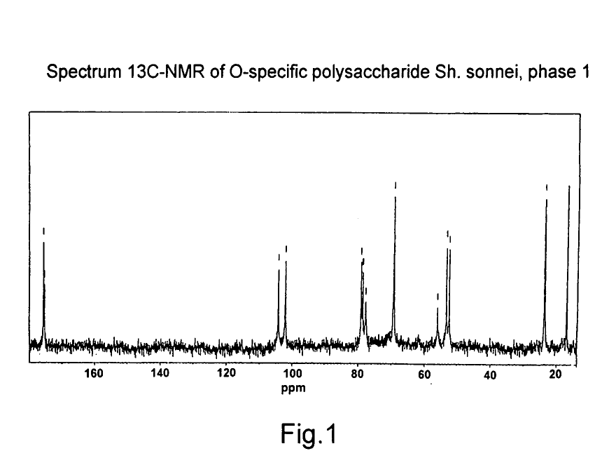

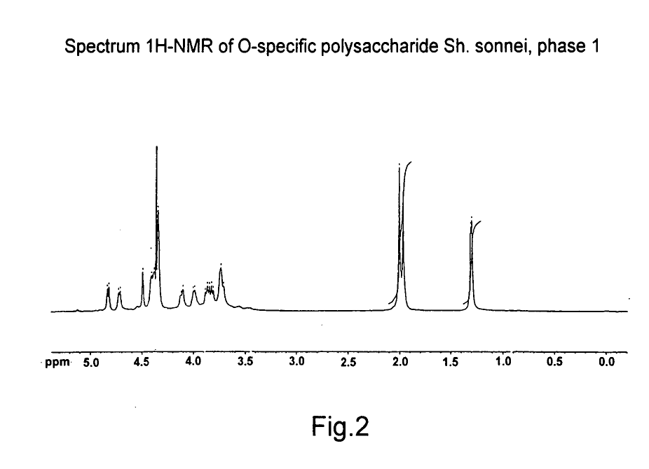

The spectra of ¹³C-NMR and ¹H-NMR O-specific polysaccharide (FIGS. 1 and 2, respectively) were recorded with the use of a Brucker WM-250 device in a D₂O solution (the sample prior to record ¹H-NMR-spectra was lyophilized from D₂O) at a temperature of 297° K. or 303° K.

An analysis of the spectral data obtained according to the COSY, TOCSY and ROESY methods allowed to make an unambiguous attribution of all the signals in the ¹³C-NMR and ¹H-NMR spectra and confirm that the repeating unit of PS is β-(1-3)-linked disaccharide 2-acetamido-2-deoxy-4-O-(2-acetamido-4-amino-2,4,6-trideoxy-β-D-galactopyranosyl)-L-altopyranosyluronic acid.

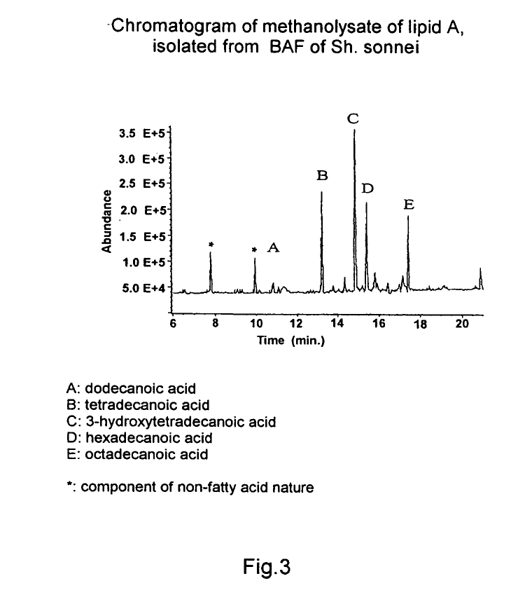

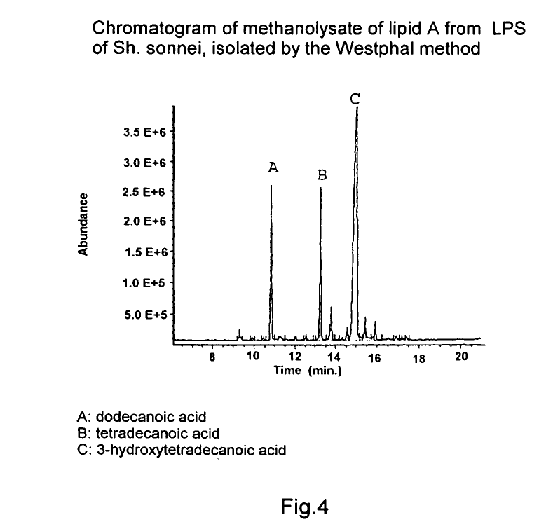

The fatty acid composition of lipid A from BAF of Sh. sonnei was established by GLC MS method with the use of methyl ethers of fatty acids formed from lipid A after methanolysis (1M HCl in MeOH, 100° C., 6 hours). Chromatograms of lipid A methanolysate isolated from BAF of Sh. sonnei, and lipid A from LPS isolated by the Westphal method, are presented on FIGS. 3 and 4, respectively.

### Example 3

### Preparation of a Conjugate of BAF from Sh. sonnei and Vi Antigen Salmonella enterica sv typhi

Twenty mg of 1-ethyl-3-(3-dimethylaminopropyl)carbodimide were added to a solution of 20 mg of Vi-antigen in 2 ml of water while stirring at room temperature for 10 minutes, maintaining pH to about 5.0 by adding 0.1M HCl. After 30 minutes of stirring at pH=5.0, a solution of 5 mg of BAF from Sh. sonnei was added to the reaction mixture. The reaction mixture was stirred during 16 h at a temperature of 10-12° C., dialyzed for 72 hours against distilled water and lyophilized. The obtained product was subjected to gel chromatography on a column (1×100) with Sephacryl S1000 (the limit of exclusion >8×10⁸ D) in 0.2M NaCl. The fractions eluted near the void volume of the column (yield after dialysis 7 mg) were active in RTPGA with sera against LPS of Sh. sonnei, phase I and Vi-antigen S. typhi. The starting antigens were eluted from the column at a greater holding time. The data presented above indicated the formation of a high-molecular conjugate, including both of the antigens connected by amide bonds, which makes it possible to use the BAF as a matrix for combining antigens of different nature. The possibility of modifying the BAF with the aid of different spacer groups and, thus, creating “bridge” structures binding the macromolecules of the antigens appears within the limits of this approach.

### Example 4

### Preparation of an Associate of BAF from Sh. sonnei with Human Serum Albumin

The possibility of forming an associate of BAF from Sh. sonnei with proteins was determined by gel-chromatography.

0.5 mg of BAF from Sh. sonnei was dissolved in 1 ml of 0.9% NaCl, the obtained solution was heated at 60° C. for 15 minutes and subjected to gel-chromatography on a column with Sepharose 4B-CL, equilibrated by 0.9% NaCl. It was shown that BAF from Sh. sonnei was eluted from a column near a void volume. Human serum albumin (HSA) under these conditions was eluted with Kd=0.67, choleric anatoxin—with Kd=0.85. In order to produce the associate, a solution of BAF from Sh. sonnei (concentration of 10 mg/ml) was mixed with an equal volume of a HSA solution (concentration of 1 mg/ml) and incubated at 60° C. for 15 minutes. The obtained mixture was applied onto a column (1×100 cm) with Sepharose 4B-CL, equilibrated by 0.9 % NaCl. The formation of the associate was judged by the complete absence of a peak in the region Kd=0.67 and by the presence of a single peak eluting with the void volume of the column. Similar results were obtained during an analysis of the associate of BAF from Sh. sonnei and the choleric anatoxin.

Thus, the capability of forming associates in which there is no covalent bond between the macromolecules of the antigens of lipopolysaccharide and protein nature opens the possibility for use of BAF as a crrier for protein antigens.

### Example 5

### Pyrogenicity Determination of BAF Preparations in Experiments on Rabbits

The pyrogenicity of preparations of BAF from Sh. sonnei, phase 1, Sh. flexneri, Sh. dysenteriae, type 1, S. enterica sv typhi, Escherichia coli 055 was determined in comparison with samples of LPS obtained by the Westphal method from the same strains of endotoxic microorganisms. The test was carried out on rabbits in accordance with the requirements of State Pharmacopoeia XII ed. and European Pharmacopoeia, USP. Determination of the pyrogenicity was carried out on 3 rabbits of the Shinshill breed having a weight of 2.8-3.05 kg. After administration of the preparation, the rectal temperature of the rabbits was measured three times at an interval of 1 hour. A preparation was considered to be apyrogenic if the sum increase of temperature (on three rabbits) did not exceed 1.15° C. The results of measurement of the temperature are presented in Table 2.

It follows from the data of Table 2 that all the LPSs isolated by the Westphal method have a high level of pyrogenicity, while the BAFs proposed in the present invention in a dose of 0.05 μg per kg of rabbit body weight do not cause a pyrogenic effect in rabbits. According to the level of pyrogenicity they correspond to the requirements of the Technical Committee of Experts of WHO for polysaccharide vaccines.

TABLE 2
Pyrogenicity of BAF preparations

| | Temperature | Result of |
|---|---|---|
| | measurement | determination of |
| Preparation | data | level of pyrogenicity |
| Vaccine VIANVAC¹⁾ | (0., 0.3, 0.3) Σ 0.7 | Apyrogenic |
| Vaccine TYPHIM Vi²⁾ | (0.2; 0.5, 0.2) Σ 0.9 | Apyrogenic |
| BAF Sh. flexneri 2a | (0.5, 0.2, 0.4) Σ 1.1 | Apyrogenic |
| BAF Sh. sonnei, phase 1 | (0.5, 0.2, 0.3) Σ 1.0 | Apyrogenic |
| BAF Sh. dysenteriae, type 1 | (0.2, 0.5, 0.3) Σ 1.0 | Apyrogenic |
| BAF Salmonella enterica sv | (0.3, 0.3, 0.5) Σ 1.1 | Apyrogenic |
| typhi | | |
| BAF Escherichia coli | (0.2, 0.4, 0.3) Σ 0.9 | Apyrogenic |
| LPS Sh. flexneri 2a | (0.8, 0.6, 0.8) Σ 2.2 | Highly-pyrogenic |
| LPS Sh. dysenteriae, type 1 | (1.3, 1.2, 1.0) Σ 3.6 | Highly-pyrogenic |
| LPS Escherichia coli (Sigma) | (1.5, 1.0, 1.0) Σ 3.5 | Highly-pyrogenic |
| LPS Sh. sonnei, phase 1 | (1.4, 1.0, 0.9) Σ 3.3 | Highly-pyrogenic |
| LPS Salmonella enterica sv | (1.6, 1.1, 0.9) Σ 3.6 | Highly-pyrogenic |
| typhi (Sigma) | | |

¹⁾Commercial sample of the Vi-antigenic polysaccharide typhoid vaccine of the “Gritvak” enterprise, Russia (international trade mark - ATV-D-TEAM).
²⁾Commercial sample of the Vi-antigenic polysaccharide typhoid vaccine of the “Pasteur-Merier” firm, France.

### Example 6

### Determination of the Endotoxicity of BAF Preparations in Experiments on D-Galactosamine Sensitized Mice

Assessment of the endotoxicity was carried out on a special model in vivo on outbred mice sensitized by D-galactosamine (D-GalN)—a hepatotoxic agent sharply increasing the sensitivity of mice to the production of the endogenic tumor necrosis factor (TNF), induced by LPS. The sensitivity of an organism to toxic products of macrophages is increased without modification of the real causa for endotoxicity—the reactivity of the macrophages in response to LPS. Fifteen mg of D-GalN together with a sample of BAF or LPS, obtained in accordance with the Westphal method, were administered peritoneally to mice having a weight of 18-20 grams. The mice survival in groups of 5 animals was determined in the course of 48 hours (see Table 3). Complete survival of the mice was registered when there was administration of BAF preparations in the following dose ranges: BAF from Sh. sonnei phase 1-100 μg, BAF from Sh. flexneri—1 μg, BAF from Sh. dysenteriae type 1-10 μg, BAF from Salmonella enterica sv typhi—1 μg. The survival of mice with the administration of LPS preparations obtained according to the Westphal method was noted in a dose range that was 1000 times less as compared with BAF.

TABLE 3
Determination of the endotoxicity of LPS preparations when
administered to outbred white mice sensitized with D-GalN

| | Survival mice after administration of 15 mg of | | | | | | |
|---|---|---|---|---|---|---|---|
| | D-GalN + preparation in a dose of (μg) | | | | | | |
| Preparation | 0.001 | 0.01 | 0.1 | 1.0 | 10 | 100 | 400 |
| BAF Sh. flexneri 2a | ND | ND | 100% | 100% | 0% | 0% | 0% |
| BAF Sh. sonnei | ND | ND | 100% | 100% | 100% | 100% | 60% |
| BAF Sh. dysenteriae | ND | ND | 100% | 100% | 100% | 0% | 0% |
| type 1 | | | | | | | |
| BAF Salmonella | ND | 100% | 100% | 100% | 0% | 0% | 0% |
| enterica sv typhi | | | | | | | |
| LPS Sh. sonnei | 0% | 0% | 0% | 0% | 0% | ND | ND |
| LPS S. Typhimurium | 0% | 0% | 0% | 0% | 0% | ND | ND |
| (Sigma, U.S.A.) | | | | | | | |

### Example 7

### Adverse Reactions Under Clinical Application BAF on Adult Volunteers

A preparation of BAF from Sh. sonnei as a shigellosis candidate-vaccine in doses of 25, 50, 75 μg in a phenol-phosphate buffer as a solvent was administered once, twice and three times at an interval of 3-4 weeks subcutaneously to 1765 adult volunteers into the upper third of a shoulder under the guidance of the National Control Authorities of the Ministry of Health of the Russian Federation. The appearance of toxic reactions which related to three groups: symptoms of endotoxic shock, general adverse reactions, local adverse reactions, was registered during 72 hours.

Symptoms of endotoxic shock: tachycardia, blood pressure down, sharp increase of temperature, were not noted in any of the 1765 volunteers in the age group of from 17 to 65 years, who received the preparation once or a multiple number of times.

General adverse reactions: fatigue, headache, nausea, diarrhea, rhinitis, stomach ache, chill, vertigo, kept track of in 129 volunteers in two controlled clinical experiments with the use of placebo, were not registered after one or two administrations with doses of 25, 50 and even 75 μg.

Insignificant local adverse reactions - pain and redness were detected in 7.47% and 8.3% of the cases, respectively in vaccines and placebo group. Such local reactions as the occurrence of infiltrates, abscesses, lymphangitis, pain or an increase of regional lymph nodes were not detected.

Thus, the complex of adverse reactions was judged to be mild.

### Example 8

### Temperature Reactions Under of Clinical Application of BAFs on Adult Volunteers

A preparation of BAF from Sh. sonnei was administered once, twice or three times at an interval of 3-4 weeks subcutaneously to 129 adult volunteers in the upper third part of the shoulder as a shigellosis candidate-vaccine in doses of 25, 50, 75 μg in a phenol-phosphate buffer as a solvent. In the course of 72 hours, the occurrence of temperature reactions, which related to three groups—weak <37.5° C., middle 37.6-38.5° C., strong >38.6° C., was registered. Strong and middle temperature reactions were not registered in any of the volunteers in the case of all the used schemes and doses for immunization. A weak increase of body temperature to 37.5° C. was observed relatively rarely (not more than 5-10% of those inoculated), and this parameter significantly did not differ from the number of temperature reactions in the placebo group.

### Example 9

### Adverse and Temperature Reactions with Clinical use of BAFs on Children

A preparation of BAF from Sh. sonnei was administered once and twice at an interval of 4 weeks subcutaneously into the upper third of a shoulder as a shigellosis candidate-vaccine in doses of 25 and 50 μg in a phenol-phosphate buffer to 35 children from 2.8 to 6 years old, to 21 children from 7 to 10 years old, to 18 children from 10 to 14 years old under the guidance of the National Control Authorities of the Ministry of Health of the Russian Federation.

Symptoms of endotoxic shock—acute tachycardia, blood pressure down, sharp rise of temperature, were not observed in any of the 74 children in the age groups of from 2.8 to 14 years old, who were given the preparation once or twice.

General adverse reactions—fatigue, headache, nausea, diarrhea, rhinitis, stomach ache, chill, vertigo—were not registered either (Table 4).

Insignificant local adverse reactions (pain) were observed in 2.7% of the cases.

Temperature reactions in the form of a strong and average increase of body temperature were not observed in children in any of the used schemes and doses of immunization. A weak increase of body temperature to 37.5° C. was observed in 5.4% of the inoculated.

TABLE 4
General and local adverse reactions after subcutaneous immunization of
children with BAF from Sh. sonnei.

| | | Frequency of |
|---|---|---|
| | | registration of adverse reactions in |
| | | children immunized with BAF from |
| Adverse reactions | | Sh. sonnei (74 persoms) |
| TEMPERATURE | | |
| Increase of temperature | Abs. | 4 |
| to 37.1-37.5° C. | (%) | 5.4% |
| Increase of temperature | Abs. | 0 |
| to 37.6-38.5° C. | (%) | 0 |
| Increase of temperature | Abs. | 0 |
| to 38.6 and more ° C. | (%) | 0 |
| LOCAL | | |
| Redness | | 0 |
| Induration | | 0 |
| Pain at place of | Abs. | 2 |
| administration | | |
| of preparation | (%) | 2.7% |

| | |
|---|---|
| GENERAL | |
| Nausea | 0 |
| Diarrhea | 0 |
| Headache | 0 |
| Rhinitis | 0 |
| Vertigo | 0 |
| Fatigue | 0 |
| Chill | 0 |

The complex of adverse reactions is evaluated as “weak.”

### Example 10

### Induction of the Tumor Necrosis Factor in Volunteers with Subcutaneous Administration of Vaccine Doses of a BAF Preparation

A study was carried for investigation of the production of the tumor necrosis factor (α-TNF) 3 hours after the first and after the second deep subcutaneous immunization with BAF from Sh. sonnei in doses of 50 and 75 μg and it was compared with the background level. The content of α-TNF was determined with the aid of the test system “Pro Cont-TNF” and expressed in pg/ml.

A statistically significant increase of the level of α-TNF—the main mediator of the endotoxic reaction—was not detected, even under immunization with the maximal dose of 75 μg (Table 5).

TABLE 5
Low induction of α-TNF in volunteers after immunization with the BAF
preparation.

| Dose of BAF | | α-TNF, pg | | | |
|---|---|---|---|---|---|
| Sh. sonnei, | | Prior to | 3 hours | Prior to | 3 hours after |
| μg | N* | vaccination | after vaccination | revaccination | revaccination |
| 50 | 10 | 208.42 ± 53.82 | 150.76 ± 35.98 | 118.82 ± 23.99 | 143.49 ± 16.66 |
| 75 | 12 | 172.41 ± 49.44 | 244.31 ± 74.17 | 79.13 ± 12.58 | 140.02 ± 38.03 |

N* - number tested paired sera obtained from volunteers.

### Example 11

### An Analysis of the Serological Activity of O-Antigenic Determinants in BAF Preparations

The serological activity of Sh. sonnei, Sh. flexneri 2a, Sh. dysenteriae type 1, Salmonella enterica sv typhi, Escherichia coli 055 BAF preparations was determined in a inhibition passive hemagglutination reaction (IHA) with use of a corresponding commercial erythrocytic diagnosticum (MSRIEM, Russia) and monoreceptor O-serum of Sh. sonnei, Sh. flexneri 2a, Sh. dysenteriae type 1, S. enterica sv typhi, E. coli 055 (SPbNIIVS, Russia; MSRIEM, Russia; Diagnostic Pasteur, France). Samples of BAF or LPS preparations, prepared in accordance with the Westphal method in a concentration of 50 μg/ml were sequentially introduced into cups. The concentration of the corresponding antiserum was brought to 4 SU (serologic units). The minimum concentration causing inhibition of the reaction after addition of erythrocytic diagnosticum—inhibition point—was expressed in μg/ml.

The inhibition points for different samples of BAF and LPS are presented in Table 6. The serological activity of BAFs did not differ from such for corresponding LPSs. Thus, in the process of preparing the BAF described in Example 1 of the instant invention, there was no change in the structure of O-specific polysaccharide chains, and their antigenic activity remained high.

TABLE 6
The concentration of IHA for different samples of Sh. sonnei,
Sh. flexneri 2a, Sh. dysenteriae type 1, Salmonella enterica sv typhi,
Escherichia coli 055 BAFs.

| Sample | Inhibition concentration, μg/ml |
|---|---|
| BAF Salmonella enterica sv typhi | 1.56 |
| LPS Salmonella enterica sv typhi | 1.56 |
| O-PS Salmonella enterica sv typhi | 12.5 |
| BAF Escherichia coli 055 | 3.12 |
| LPS Escherichia coli 055 | 1.56 |
| BAF Sh. flexneri 2a | 1.56 |
| LPS Sh. flexneri 2a | 0.78 |
| BAF Sh. dysenteriae type 1 | 0.37 |
| LPS Sh. dysenteriae type 1 | 1.56 |
| BAF Sh. sonnei | 3.125 |
| O-PS Sh. sonnei | 25 |
| LPS Sh. sonnei | 3.125 |

### Example 12

### Activation of Humoral Immune Response with BAF Preparations in Experiments on Laboratory Animals

In order to determine the induction of a humoral immune response, mice (CBAXC57B1/6). Fl were intraperitoneally immunized with a series of preparations of Sh. sonnei, Sh. flexneri 2a, Sh. dysenteriae type 1 BAF in doses of 400, 100, 10, 1 and 0.1 μg per mouse. In another experiment the mice were immunized with a series of preparations of Salmonella enterica sv typhi, Escherichia coli 055 BAF in doses of 1, 10, 50 μg per mouse. After 15 and/or 30 days peripheral blood serum was taken from the animals and the level of O-specific antibodies was determined in an ELISA test and IHA. The BAF preparations stimulate an immune response after a single administration, the titer of anti-O antibodies is determined on the 15th day, wherewith the maximum immunogenic effect was observed within the range of doses from 10 to 50 μg per mouse. A significant amount of antibodies of the main classes IgG, IgM, IgA against homologous LPS was determined in the peripheral blood.

### Example 13

### Induction of an Adaptive Immunity to Typhoid Feverwith Immunization of Laboratory Animals with BAF from Salmonella enterica sv typhi

Mice (CBAXC57B1/6) Fl were intraperitoneally immunized with a preparation of BAF from S. enterica sv typhi and LPS from S. enterica sv typhi, obtained according to the Westphal method, in doses of 25, 5, 1, 0.2, 0.04, 0.008, 0.0016 μg. A physiological solution was administered to a control group of animals. After 12 days the groups of animals were infected with a dose of 1×10³ cells (50 LD₅₀) of a virulent typhoid strain Ty2 No.4446 in a sterile physiological solution containing 5% mucin. The survival of the animals in the groups were registered during 7 days. Both the highly endotoxic LPS and the low endotoxic BAF induced an adaptive immunity to typhoid fever and provided a high level of protection for the animals (80-100%) against challenge in the case of administration in doses of 25-5 pg (see Table 7). All the animals in the control group died. ED₅₀ BAF—0.037 μg; ED₅₀—LPS—0.081 μg.

TABLE 7
Induction of adaptive immunity to typhoid fever after immunization of
laboratory animals with BAF from Salmonella enterica sv typhi.

| | Survival of mice | |
|---|---|---|
| | immunized with BAF | |
| | S. enterica sv typhi | Survival of mice immunized |
| Immunization | and infected by | with LPS S. enterica sv typhi |
| dose | 1000 cells of strain | and infected by 1000 cells of |
| (μg) | S. enterica sv typhi Ty2 | strain S. enterica sv typhi Ty2 |
| 25 | 10/10 | 8/10 |
| 5 | 8/10 | 7/10 |
| 1 | 5/10 | 6/10 |
| 0.2 | 8/10 | 6/10 |
| 0.04 | 6/10 | 4/10 |
| 0.008 | 5/10 | 6/10 |
| 0.0016 | 2/10 | 4/10 |
| — | 0/10 | 0/10 |

### Example 14

### Induction of Mucosal Immunity Against Infection Sh. sonnei After Systemic Immunization of Laboratory Animals with BAF from Sh. sonnei (Sereny Test)

Guinea pigs having a weight of 200-250 g were immunized subcutaneously with BAF from Sh. sonnei in doses of 25, 50 μg into the back region twice at an interval of 10 days. A physiological solution was injected to control animals instead of the preparation. Ten days after the last immunization, a suspension of cells of a virulent strain Sh. sonnei 5063 in a dose close to ID₁₀₀ (2×10⁹ cells) and in a dose close to 2ID₁₀₀ (4×10⁹ cells) in 30 μl of a sterile physiological solution was administered into the eye conjunctiva. Shigella keratoconjunctivitis developed in all the animals of the control group 5 infected by a dose of 4×10⁹ cells and in 75% of animals of the control group 6 infected by a dose of 2×10⁹ cells (see Table 8). Double immunization with a dose of 50 μg provided protection to 55% of the eyes of animals in the case of infection by a dose of 4×10⁹ cells and 75% protection of eyes of animals in the case of infection by a dose of 2×10⁹ cells. Two-time immunization with a dose of 25 μg provided protection to 75% of the eyes of animals in the case of infection by a dose of 4×10⁹ cells and 80% protection of the eyes of animals in the case of infection by a dose of 2×10⁹ cells. Thus, an expressed local anti-infection immunity was registered in the case of subcutaneous immunization by a preparation of BAF from Sh. sonnei.

### Example 15

### Activation of Systemic Immune Response by a BAF Preparation During Immunization of Adult Volunteers

A study of the BAF from Sh. sonnei immunogenicity as a Sh. sonnei candidate-vaccine was carried out under conditions of a controlled trial with immunization of adults 18-22 years old under the guidance of the National Control Authorities of the Ministry of Health of the Russian Federation. Systemic immune response to BAF from Sh. sonnei was studied by examining paired sera of venous blood obtained from patients prior to immunization and 28-30 days after immunization. Specific indirect haemagglutination (IHA) reaction was performed using a commercial kit (series 3 K 60) for serologic diagnosis of Sonnei dysentery, produced by the Gabrichevsky Enterprise Institute (Moskow). ELISA test was performed by using ELISA-test system based on LPS from Sh. sonnei to determine the serum antibodies IgG, IgA, IgM classes. Totally 380 sera obtained from patients inoculated with BAFs from Sh. sonnei and a placebo were studied.

An 18.5 times increase of the GM titers of agglutinating, 5.9 times IgG, 2.6 times IgM, 19.3 times IgA increase of specific anti-LPS Sh. sonnei antibodies were after vaccination of persons with BAF from Sh. sonnei (Table 9). The GM titer in the group of persons receiving the placebo, prior to and after vaccinations, did not differ in a statistically significant manner.

The percentage of persons with seroconversions (four-time and more increase of the antibodies) by the in the group inoculated with BAFs from Sh. sonnei a month after the vaccinations was 94.7% for agglutinating antibodies, 71.6% for IgG, 51% for IgM, 77% for IgA anti-O-antibodies.

The combination of clinical-immunological data shows the high level of activation of systemic humoral immune response to BAF from Sh. sonnei in the volunteers, especially its IgA class.

TABLE 8
Induction of mucosal immunity against Sh. sonnei infection after systemic immunization of laboratory
animals with BAF from Sh. sonnei (Sereny test).

| | | | | | | Number of | |
|---|---|---|---|---|---|---|---|
| | | | | | Number of | eyes protected | |
| | Vaccine | Infection | Infected | Infected | eyes with | against | Percentage of |
| No. | dose | dose | animals | eyes | keratoconjunctivitis | keratoconjunctivitis | protected eyes |
| 1 | 50 μg. | 0.4 × 10¹⁰ | 10 | 20 | 9 | 11 | 55% |
| | 50 μg | cells/eye | | | | | |
| 2 | 50 μg. | 0.2 × 10¹⁰ | 10 | 20 | 5 | 15 | 75% |
| | 50 μg | cells/eye | | | | | |
| 3 | 25 μg. | 0.4 × 10¹⁰ | 10 | 20 | 5 | 15 | 75% |
| | 25 μg | cells/eye | | | | | |
| 4 | 25 μg. | 0.2 × 10¹⁰ | 10 | 20 | 4 | 16 | 80% |
| | 25 μg | cells/eye | | | | | |
| 5 | Control | 0.4 × 10¹⁰ | 10 | 20 | 20 | 0 | 0% |
| | | cells/eye | | | | | |
| 6 | Control | 0.2 × 10¹⁰ | 10 | 20 | 15 | 5 | 25% |
| | | cells/eye | | | | | |

TABLE 9
Evaluation of systemic humoral immune response against BAF from Sh. sonnei
a month after immunization of volunteers

| Multiplicity of growth of IgA anti-O antibodies | | 19.3 | — |
|---|---|---|---|
| Geometrical mean titers of IgA | 4 weeks after vaccination | 1235 ± 168 | 58 ± 9 |
| anti-O antibodies | | (75) | (80) |
| (Number of studied pairs of serum) | Prior to vaccination | 64 ± 7.5 | 83 ± 17 |
| | | (75) | (80) |
| Multiplicity of growth of IgM anti-O antibodies | | 2.62 | 1.45 |
| Geometrical mean titers of IgM | 4 weeks after vaccination | 249 ± 72 | 153 ± 28 |
| anti-O antibodies | | (78) | (81) |
| (Number of studied pairs of serum) | Prior to vaccination | 95 ± 29 | 105 ± 33 |
| | | (78) | (81) |
| Multiplicity of growth of IgG anti-O antibodies | | 5.9 | — |
| Geometrical mean titers of IgA | 4 weeks after vaccination | 940 ± 150 | 131 ± 16 |
| anti-O antibodies | | (78) | (81) |
| (Number of studied pairs of serum) | Prior to vaccination | 181 ± 40 | 138 ± 22 |
| | | (78) | (81) |
| Multiplicity of growth of agglutinating antibodies | | 18.5 | — |
| Geometrical mean titers of IgA | After vaccination | 724 | 42 |
| anti-O antibodies (GMTA) | | (95) | (80) |
| (Number of studied pairs of serum) | Before vaccination | 39 | 34 |
| | | (95) | (80) |

| | | |
|---|---|---|
| Name of preparations | BAF from | Placebo |
| | Sh. sonnei | |

### Example 16

### Activation of the Mucosal Immune Response by a BAF Preparation in the Case of Immunization of Adult Volunteers

The presence of specific secretory antibodies against LPS were studied in pair samples of the saliva of volunteers (30 persons), obtained prior to and 4 weeks after immunization with BAF from Sh. sonnei. All the saliva samples and coprofiltrates were encoded, combined into blocks and stored at a temperature of minus 18-20° C. prior to testing.

The IHA and ELISA methods with the use of monoclonal antibodies against the secretory component IgA (clone GA) and against the a-chain IgA were used to determine the antibodies.

In the IHA, a statistically significant increase of the indexes of titers of antibodies in the saliva was observed after vaccination, wherewith, in 72% of the persons, a 4-time conversion of antibodies in the saliva was observed, and the multiplicity of the increase of the titer of antibodies was 4.21. In the ELISA, a statistically significant increase of the geometrical mean titer for O-specific IgG, IgA antibodies was detected, and also for IgA with a secretory component (sIgA), tested in saliva; the multiplicity of the increase of the antibody titer was respectively 5.47, 4.37 and 4.0 (Table 10).

TABLE 10
GM titers detected in the saliva of volunteers prior to and after
immunization with BAF from Sh. sonnei.

| | Number of | GM titers of | | |
|---|---|---|---|---|
| | studied | anti-LPS antibodies | | |
| | saliva | Prior to | 4 weeks after | Multiplicity of |
| Antibodies | samples | vaccination | vaccination | increase |
| Agglutinating | 30 | 2.4 | 10.1 | 4.21 |
| IgG | 30 | 9.2 | 50.3 | 5.47 |
| IgA | 30 | 358 | 1563 | 4.37 |
| sIgA | 30 | 18.2 | 72.8 | 4.0 |

### Example 17

### Activation of Systemic Immune Response by a BAF Preparation After Immunization of Children

Three groups of children were formed by the method of chance selection (the unit of selection—1 child): the first group of 35 children was formed of children in the age group of 2.8-6 years; the second group of 21 children was formed of children in the age group of 7-10 years; the third group of 18 children was formed of children in the age group of 11-14 years. Sixty nine children were immunized subcutaneously with a preparation of BAF from Sh. sonnei in a dose of 50 μg, with the exception of five children in the age group of 2.8-4 years who were given a dose of 25 μg. The systemic O-LPS specific immune response was studied by studying pair portions of serum of venous blood obtained from 41 persons inoculated prior to immunization and 14 days after immunizations, IHA tests, and ELISA, using a commercial set (series 3 K 60) for diagnosis of Sonne shigellosis, which is produced by the Gabrichevsky Institute, and the ELISA test system.

TABLE 11
Evaluation of the systemic immune response to BAF from Sh. sonnei 14 days after immunization of children of three
age groups.

| | | GM titer of | | | | | | | |
|---|---|---|---|---|---|---|---|---|---|
| | | agglutinating anti- | | GM titer of IgG anti- | | GM titer of IgM anti- | | GM titer of IgA anti- | |
| | | LPS antibodies | | LPS antibodies | | LPS antibodies | | LPS antibodies | |
| | Number | | 2 weeks | | 2 weeks | | 2 weeks | | 2 weeks |
| Groups of | of tested | Prior to | after | Prior to | after | Prior to | after | Prior to | after |
| vaccinated | serums | vaccination | vaccination | vaccination | vaccination | vaccination | vaccination | vaccination | vaccination |
| Children | 14 | 141 ± 33 | 1187 ± 326 | 44 ± 8.5 | 168 ± 88 | 138 ± 42 | 476 ± 93 | 42 ± 3 | 411 ± 109 |
| 2.8-6 | | | | | | | | | |
| years | | | | | | | | | |
| old | | | | | | | | | |
| Children | 9 | 323 ± 93 | 2389 ± 519 | 74 ± 18 | 435 ± 262 | 217 ± 72 | 507 ± 103 | 44 ± 4 | 1164 ± 304 |
| 7-10 | | | | | | | | | |
| years old | | | | | | | | | |
| Children | 18 | 299 ± 108 | 2580 ± 332 | 71 ± 16 | 593 ± 144 | 186 ± 26 | 419 ± 61 | 58.7 ± 12 | 1280 ± 397 |
| 11-14 | | | | | | | | | |
| years | | | | | | | | | |
| old | | | | | | | | | |
| Children | 41 | 235 ± 55 | 1947 ± 240 | 61 ± 9 | 360 ± 95 | 174 ± 24 | 456 ± 47 | 48.9 ± 6 | 673 ± 216 |
| 2.8-14 | | | | | | | | | |
| years | | | | | | | | | |
| old | | | | | | | | | |

The percent of 4-fold seroconversion in all the groups of children receiving BAF from Sh. sonnei, 14 days after vaccination was for the agglutinating, IgG, IgM, IgA antibodies respectively 90.2%, 73%, 48.7%, 92.7%. In the first group of vaccinated children (2.8-6 years old) the percent of 4-fold seroconversion was for agglutinating, IgG, IgM, IgA antibodies respectively 100%, 57%, 71%, 85.7%, in the second group of children (7-10 years old)—71.4%, 66.7%, 44.4%, 85.7%, and in the third group (11-14 years old)—89.5%, 88.7%, 33.3%, 100%.

The geometrical mean titers of anti-O agglutinating antibodies (GM titer) are significantly greater after vaccination in the groups of children inoculated with BAF from Sh. sonnei, as compared with the background level (Table 11). The GMTA of IgG antibodies prior to immunization in the vaccinated groups was 44, 74, 71 respectively in the 1st, 2nd and 3rd groups (Table 11), and 14 days after vaccination 168, 435 and 593 respectively, while the multiplicity of the increase of the titer of the antibodies—3.8, 5.9 and 8.9. The increase of the level of IgM anti-O antibodies among those immunized with the preparation of BAF from Sh. sonnei was less, but nevertheless, statistically significant in all the groups of those immunized. The expressed induction by BAF from Sh. sonnei of anti-O IgA antibodies, which play a key role in immunity against dysentery, should be noted. The more significant increase of IgA antibodies was observed, in particular, prior to immunization GMTA was 42, 44, 58.7, and after 411, 1164, 1280, while the multiplicity of the increase of the titer of the antibodies was 6.6; 17.3; 21.7 times (Table 11).

### Example 18

### Induction of Tolerance to Endotoxin by BAF Preparations

In order to determine the induction of tolerance to lipopolysaccharide, mice (CBAXC57B 1/6) F1 (8 mice per group) were immunized intraperitoneally with preparations of BAF from Sh. sonnei, Salmonella enterica sv typhi, Escherichia coli 055 in doses of 0.1, 1.0, 10.0 μg per mouse. LPS from SH. sonnei, administered in those same doses, and an apyrogenic physiological solution were used for control. Twelve hours later, in order to create a model of endotoxic shock, the animals were immunized with the LPS preparation in a dose of 0.1 μg per mouse intraperitoneally together with 15 μg of D-galactosamine. This dose of LPS was earlier determined to be absolutely lethal (100 LD₅₀). In the groups of animals immunized with BAF from Sh. sonnei, S. enterica sv typhi, E. coli 055, and also LPS Sh. sonnei, the survival of the animals was 100% as result of the induction of tolerance. In the control group receiving the physiological solution, all the animals died within 24 hours. Thus, the low endotoxic BAFs from Sh. sonnei, S. enterica sv typhi, E. coli 055, like the classical LPS from Sh. sonnei, had an expressed tolerogenic effect within the range of doses from 0.1 to 10 μg.

### Example 19

### Induction of Early γ-Interferon in vivo by a BAF Preparation

In order to study the production of γ-interferon, mice (CBAXC57B1/6) were intraperitoneally immunized with a preparation of BAF from Sh. sonnei and LPS from Sh. sonnei in doses of 10 and 100 μg. Serum of peripheral blood in an earlier selected peak point was taken from the animals after 7.5 hours. The concentration of γ-interferon was determined with use of the OptEIA™ Mouse interferon-γ test-system (Pharmagen). As is evident from Table 12, the BAF preparations caused an increase of the concentration of γ-interferon in the serum. In respect to BAF from Sh. sonnei, an increase of the production of γ-interferon, dependent on the dose, was noted.

TABLE 12
Controlled induction of early γ-interferon in vivo by a BAF preparation.

Concentration of
γ-interferon, pg/ml

| Preparation | 10 μg/mouse | 100 μg/mouse |
|---|---|---|
| LPS from Sh. sonnei | 541 ± 158 | 1039 ± 728 |
| BAF from Sh. sonnei | 55 ± 50 | 407 ± 253 |
| Control | 25 ± 25 | 50 ± 48 |

### Example 20

### Induction of Resistance in Mice to Staphylococcus aureus Infection by a Preparation of BAF from Shigella sonnei, Salmonella enterica sv typhi, Escherichia coli 055

In order to detect the induction of resistance to Staphylococcus aureus, strain 209, white mice were immunized intraperitoneally by a preparation of BAF from Sh. sonnei in a dose of 100 μg/mouse (equivalent to a human dose), and after 7 days infected with 5×10⁶ microbial cells of S. aureus, strain 209. On the 1st, 2nd, 3rd, 4th and 10th day mice of the test and control (intact) groups were dissected (5 from each group on each of the aforesaid days). Seedings were taken from the blood, liver, spleen, mesentery and kidneys onto a solid nutrient medium, and in the following days the colonies were typed. The index of dissemination was determined—the ratio of positive seedings to the sum number of samples. The dynamics of the index of dissemination served as an index of the nonspecific resistance of mice to Staphylococcus aureus (Table 13).

TABLE 13
Dynamics of the index of dissemination of internal organs of mice.

Index of dissemination

| | Test | |
|---|---|---|
| | 100 μg of BAF from | |
| Term (days) | Sh. sonnei | Control |
| 1 | 0.91 | 1.0 |
| 2 | 0.48 | 0.93 |
| 3 | 0.20 | 0.75 |
| 6 | 0 | 0.12 |
| 10 | 0 | 0.05 |

Thus, the preparation of BAF from Sh. sonnei in a dose of 100 μg stimulated nonspecific resistance to staphylococcus infection.

### Example 21

### Induction of Resistance in Mice to Infection by Virus of Influenza A, Stain PRA-8 with the Aid of BAF from Shigella sonnei, Salmonella enterica sv typhi, Escherichia coli 055

In order to study the induction of resistance to a viral infection by the action of BAF, mice (CBA line, males weighing 18-20 g, groups with 10 animals in a group) under the action of a light ether narcosis were infected intemasaly by 1 DCL of the virus of influenza A, strain PRA-8. BAFs from Sh. sonnei, S. enterica sv typhi, E. coli 055 were administered subcutaneously in a a dose of 100 μg 2 days prior to infection. The BAF preparations caused 80-90% survival of the mice (observation was carried on for 10 days) with a 85-90% death rate of the animals in the control groups. Thus, in the model described above, the BAFs exhibited an expressed prophylactic action.

### Example 22

### Anti-Cancer Activity of a BAF Preparation Upon Infection of DBA/2 Mice by Grafted Cells of Mastocytoma P815

Determination of anti-cancer activity was carried out on DBA/2 mice. The mice—10 animals in a group, were immunized intraperitoneally with preparations of BAF from S. typhi in doses of 10 μg and 100 μg 24, 72 hours prior to and 12, 24 hours after injection of cells of mastocytoma P815. The injections of mastocytoma P815 were carried out into one of the rear limbs in a dose of 1×10⁵ cells. Mice of the same line were used as control, the same doses of tumor cells were administered thereto. The percent of survival of the animals during 31 days was fixed.

The two-time administration of the preparation of BAF from S. typhi in a dose of 100 μg 24 and 72 hours prior to injection with tumor cells—group II, or 12, 24 hours after administration of tumor cells—group IV, resulted in a significant increase of the length of life of these animals as compared with the group which did not receive injections of the preparation of BAF from S. typhi—group I. At the same time, a one-time injection of a preparation of BAF from S. typhi in a dose of 10 μg does not activate anti-cancer immunity and independent of the time of administration of tumor cells results in a reduction of the indexes of survival (groups III, V). Thus, the preparation of BAF from S. typhi has protective effects during the tumor growth at a dose of 100 μg per mouse administered twice (Table 14).

TABLE 14
Anti-cancer activity of a BAF preparation in the case of infection of
DBA/2 mice by grafted cells of mastocytoma P815.

| | Survival of animals, % | | | | |
|---|---|---|---|---|---|
| Day | I* | II* | III* | IV* | V* |
| 0 | 100 | 100 | 100 | 100 | 100 |
| 7 | 60 | 100 | 60 | 100 | 60 |
| 11 | — | 80 | 0 | 80 | 0 |
| 19 | 0 | 60 | 0 | 60 | — |
| 31 | 0 | 20 | 0 | 60 | 0 |

I* - Control. Administration of cells of mastocytoma P815
II* - Administration of BAF from S. typhi in a dose of 100 μg two times, 24 and 72 hours prior to administration of cells of mastocytoma P815
III* - Administration of BAF from S. typhi in a dose of 10 μg 24 hours prior to administration of cells of mastocytoma P815
IV* - Administration of BAF from S. typhi in a dose of 100 μg 12, 24 hours after administration of cells of mastocytoma P815
V* - Administration of BAF from S. typhi in a dose of 10 μg 24 hours after administration of cells of mastocytoma P815

administration of cells of mastocytoma P815

### Example 23

### Prophylactic Efficacy of a Pharmaceutical Composition on the Base of BAF from Sh. sonnei as a Vaccine Against Sonne Shigellosis

The conducted field tests of the vaccine against Sonne shigellosis, which is a pharmaceutical composition on the base of BAF, were carried out in infection endemic Romanovsky county of the Saratov region. Three thousand sixty eight people were immunized. Among them, 1802 volunteers received one subcutaneous injection of the vaccine, while 1266 received an injection of a placebo. The level of infection in the group of inoculated was 0.55 per 1000, in the placebo group—7.9 per 1000. The average index of efficacy of the vaccine was 92.9% for a six-month observation period.

The preparation provided effective protection of the civilian population in the most unfavorable summer-fall period when there is a rise of infection with Sonne shigellosis.

### Example 24

### Pharmaceutical form of BAF

The vaccine preparation is prepared in a liquid form in a volume of 0.5 ml, mixing the following components:

| | | |
|---|---|---|
| Active substance: BAF from Shigella sonnei | 0.050 | mg, |
| Sodium dihydroortophosphate | 0.052 | mg, |
| Sodium hydroortophosphate | 0.017 | mg, |
| Sodium chloride | 4.150 | mg, |
| Phenol | 0.750 | mg, |
| Distilled water | 0.5 | ml. |

The obtained preparation may be stored for 36 months at 2-8° C.

### REFERENCE SOURCES

1. Helmut Brade et al., eds. Endotoxin in Health and Disease, New York: Marcel DEKKER, 1999.

2. Konadu E Y, Lin F Y, Ho V A, Thuy N T, Van Bay P, Thanh T C, Khiem H B, Trach D D, Karpas A B, Li J, Bryla D A, Robbins J B, Szu S C. Phase 1 and phase 2 studies of Salmonella enterica serovar paratyphi A O-specific polysaccharide-tetanus toxoid conjugates in adults, teenagers, and 2- to 4-year-old children in Vietnam. Infect Immun. March 2000; 68 (3): 1529-34.

3. Ashkenazi S, Passwell J H, Harlev E, Miron D, Dagan R, Farzan N, Ramon R, Majadly F, Bryla D A, Karpas A B, Robbins J B, Schneerson R. Safety and immunogenicity of Shigella sonnei and Shigella flexneri 2a O-specific polysaccharide conjugates in children. J Infect Dis. June 1999; 179(6): 1565-8.

4. Cohen D, Ashkenazi S, Green M S, Gdalevich M, Robin G, Slepon R, Yavzori M, Orr N, Block C, Ashkenazi I, Shemer J, Taylor D N, Hale T L, Sadoff J C, Pavliakova D, Schneerson R, Robbins J B. Double-blind vaccine-controlled randomised efficacy trial of an investigational Shigella sonnei conjugate vaccine in young adults. Lancet, Jan. 18, 1997; 349 (9046): 155-9.

5. Westphal O., Lueritz O., and Bister F. Uber die Extraktion von Bakterien mit Phenol/Wasser. Z. Naturforsch. 7:148-155. 1952.

6. Galanos C, Jiao B H, Komuro T, Freudenberg M A, Luderitz O. Large-scale fractionation of S-form lipopolysaccharide from Salmonella abortus equi. Chemical and serological characterization of the fractions. J Chromatogr. May 25, 1988; 440: 397-404.

7. Mikes O et al., eds., Laboratory handbook of chromatographic and allied methods, J. Wiley, Chichester, New York, Brisbane, Toronto, 1979.

8. WHO Expert Committee on Biologic Standardization. Requirements for Vi-polysaccharide typhoid vaccine. WHO Tech. Rep. Ser. 1994; 840: 14-33.

9. Aparin P. G., Golovina M. E., Yolkina S. I., Gantcho T. V., Ershov V.I., L'vov V. L. FIRST CLINICAL INVESTIGATION OF NEW LIPOPOLYSACCHARIDES: PHASE I-II CLINICAL TRIALS OF Shigellae sonnei LPS VACClNE. Proceedings of The Third Annual Conference on Vaccine Research, Bethesda, USA, 1-2 May, 2000. Abstract S18.

10. L'vov V. L., Verner I. C., Golovina M. E., Aparin P. G. Immunobiology of low toxic & pyrogenic lipopolysaccharide from Salmonella Typhi. Medical Journal of Indonesia, October, 1998, V. 7, Suppl. 1: 247-251.

11. Aparin P. G., Golovina M. E., Ershov V. I., Gancho T. V., Shmigol V. L., Pavlova L. I., Chuprynina R. P., Yolkina S. I., Rachmanov R. S., and L'vov V. L., Systemic and mucosal (local) immune response after immunization with new type low-endotoxic LPS vaccine Shigella sonnei. Proceedings of The Fourth Annual Conference on Vaccine Research, Arlington, USA, Apr. 23-25, 2001. Abstract S6.

12. Aparin P. G., Pavlova L. I., Gorbunov M. A., Golovina M. E., Gantcho T. V., Chuprinina R. P.,, Nemirovskaya T. I., Rachmanov R. S., Ershov V. I., Kurenkova E. B., Prochnitskaya I. A., Ankudinov I. V., Yolkina S. I., and L'vov V. L. Phase III efficacy trials of LPS Shigella sonnei vaccine: seasonal protection against shigellosis Sonnei in mid-Volga region. Proceedings of The Fifth Annual Conference on Vaccine Research, Baltimore, USA, May 6-8, 2002. Abstract.

1-43. (canceled)

44. A method for obtaining from a strain of bacteria a biologically active fraction (BAF) with S-LPSs having an endotoxicity that is lower than the endotoxicity of the LPSs produced by the strain as a whole, the method comprising the steps of:
(a) providing a strain of gram negative bacteria comprising a plurality of LPS molecules that, in combination, are highly endotoxic, said plurality of LPS molecules including (i) first LPS molecules with a lipid A component comprising a first set of fatty acids and (ii) second LPS molecules with a lipid A component comprising a second set of fatty acids, said first set of fatty acids having fatty acids other than tetradecanoic and dodecanoic acids and having a lower proportion of tetradecanoic and docdecanoic acids than said second set of fatty acids; and
(b) isolating the first LPS molecules to obtain the BAF with S-LPSs of lower endoxicity, wherein the BAF is characterized in that, in the lipid A component of the S-LPSs, a mole ratio of D-glucosamine and β-hydroxyacids selected from the group consisting of β-hydroxydecanoic, β-hydroxydodecanoic, β-hydroxytetradecanoic, and β-hydroxyhexadecanoic is approximately 2:1-4, and the mole ratio of D-glucosamine and higher fatty acids connected both by amide and by ester linkage in the lipid A component of the S-LPS is not 2:6.

45. The method according to claim 44, wherein the isolating step (b) comprises the following steps:
(I) extraction of a suspension, comprising cells and/or products of lysis thereof and/or products of viability thereof, with hot aqueous phenol according to Westphal with subsequent dialysis, separation of insoluble material, and lyophilization,
(II) purification of an intermediate product obtained in step (I) from protein and nucleic acid impurities by simultaneous treatment with ribonuclease and desoxyribonuclease and further with proteinase K and subsequent isolation of LPS by dialysis and lyophilization, and
(III) fractionation of the intermediate product obtained in step (II) by a method selected from the group consisting of hydrophobic chromatography, ultracentriffugation, fractionation by extraction with a mixture of chloroform-methanol-aqueous HCl 1:1:0.4-0.5 by volume, gel-electrophoresis in polyacrylamide gel, column chromatography, gel-penetrating chromatography, ultrafiltration and a combination thereof, and
(IV) isolation of the BAF by dialysis and/or gel-chromatography.

46. The method according to claim 45, which further comprises the step of purification of the intermediate product obtained in step (II) or (III), during which fractionation of the LPS is carried out on porous matrixes, wherein the method of fractionation is selected from the group consisting of preparative gel-electrophoresis in polyacrylamide gel, column chromatography, and ultrafiltration.

47. The method according to claim 45, wherein treatment with ribonuclease and desoxyribonuclease in step (II) is carried out at a temperature of 4-60° C. for 0.2-60 hours.

48. The method according to claim 47, wherein the treatment is carried out with desoxyribonuclease having an activity of 600-4000 units/mg and ribonuclease having an activity of 50-140 units/mg.

49. The method according to claim 45, wherein proteinase K having an activity of 1-20 units/mg is used in step (II).

50. The method according to claim 45, wherein the ultracentrifugation in step (III) is carried out at a temperature of 4-50° C., acceleration of 50000-150000 g for 0.5-24 hours.

51. The method according to claim 45, wherein column chromatography, which is selected from the group of methods including ion-exchange chromatography, hydrophobic chromatography, and gel-penetrating chromatography, is used in step (III).

52. The method according to claim 45, wherein in step (III) fractionation is carried out using gel-penetrating chromatography on porous gels in aqueous buffer eluents optionally containing chaotropic agents or detergents.

53. The method according to claim 52, wherein gel-penetrating chromatography is carried out using a column with Sephadex with elution by a buffer containing 0.05-4.0 moles of a lyophilizing buffer-forming component, at 4-50° C.

54. The method according to claim 45, wherein dialysis in steps (I) and (II) is carried out during 3-5 days.

55. The method according to claim 45, wherein lyophilization in steps (I) and (II) is carried out during 16-24 hours.

56. The method according to claim 44, wherein the strain is selected from the group consisting of Salmonella enterica sv typhi, Shigella sonnei phase 1, Shigella fexneri 2a, Shigella dysenrereae type 1 (Shiga), and Escherichia coli 055.

57. The method according to claim 44, where the BAF has a level of pyrogenicity, determined in a test on pyrogenicity of a dose of the BAF that does not exceed 25 ng per kg of rabbit weight, corresponds to a sum temperature increase of not more than 1.15° C.

58. The method according to claim 44, wherein the BAF is characterterized in that, in a clinical test for determination of a level of pyrogenicity with subcutaneous administration to adult volunteers in a dose to 75 μg, temperature reactions are manifested in no more than 20% of cases.

59. The method according to claim 44, wherein the BAF is characterized in that, in a clinical test for determination of a level of pyrogenicity with subcutaneous administration to children in a dose to 50 μg, temperature reactions are manifested in no more than 20% of cases.

60. The method according to claim 44, wherein the BAF is characterized in that the maximum tolerable dose does not exceed 150 μg in the case of a single subcutaneous administration to an adult.

61. The method according to claim 44, wherein the BAF is characterized in that it is a vaccine for mammals, including humans.

62. The method according to claim 44, wherein the BAF is characterized in that it is obtained from a Sh. sonnei culture and in the case of parenteral administration in doses of 1-100 μg it activates a specific immune response, producing systemic and secretory 1 gA antibodies, and provides seroconversion above 80%.

63. The method according to claim 44, wherein the BAF is characterized in that, in the case of parenteral administration to children in doses of 1-50 μg, it activates a specific immune response, producing systemic and secretory IgA antibodies, and provides seroconversion above 80%.

64. The method according to claim 44, wherein the BAF is characterized in that it is a vaccine carrier, capable of forming, with heterological protective natural antigens of protein and polysaccharide nature and with synthetic antigens, nontoxic complexes or conjugated compounds for enhancing the immunogenicity of such antigens for mammals, including humans.

65. The method according to claim 44, wherein the BAF is characterized in that it is safe tolerogenic anti-shock vaccine and in the case of parenteral administration induces reduction of sensitivity to the action of bacterial endotoxin in mammals.

66. The method according to claim 44, wherein the BAF is characterized in that it is an immunostimulator and has a therapeutic effect in the case of diseases and conditions requiring the stimulation of immunity in mammals, including humans.

67. The method according to claim 44, wherein the BAF is characterized in that it is an immunostimulator and has a therapeutic effect in the case of viral infections requiring the stimulation of immunity in mammals, including humans.

68. The method according to claim 44, wherein the BAF is characterized in that it is an immunostimulator and has a therapeutic effect in the case of bacterial infections requiring the stimulation of immunity in mammals, including humans.

69. The method according to claim 44, wherein the BAF is characterized in that it is an immunostimulator and has a therapeutic effect in the case of oncological diseases requiring the stimulation of immunity in mammals, including humans.

70. The method according to claim 44, wherein the BAF is characterized in that, when administered to a human in doses up to 100 μg, it does not cause the induction of endotoxic shock and adverse reactions.

71. The method according to claim 44, wherein the BAF is characterized in that it activates the production of cytokines, in particular γ-interferon in vivo.

72. The method according to claim 44, wherein the fractionation in step (III) comprises hydrophobic chromatography.
